(19) United States
(12) Patent Application Publication
(10) Pub. No.: US 2021/0379119 A1
(43) Pub. Date: Dec. 9, 2021

(54) NUTRITIONAL SUPPLEMENT TO EASE SIDE EFFECTS OF CANCER TREATMENT THERAPIES

(71) Applicant: Marivation LLC, Dothan, AL (US)

(72) Inventors: Michael Scott McAllister, Dothan, AL (US); Robert Hunter, Highlands Ranch, CO (US)

(21) Appl. No.: 17/339,662

(22) Filed: Jun. 4, 2021

Related U.S. Application Data
(60) Provisional application No. 63/034,706, filed on Jun. 4, 2020.

(51) Int. Cl. A61K 35/644; A61K 36/906; A61K 36/88; A61K 36/28; A61K 36/899; A61K 36/9066; A61K 36/54; A61K 36/61; A61K 36/67; A61K 9/00; A23L 33/115; A23L 33/105; A23L 33/00; A21D 2/16; A21D 2/36; A21D 13/28; A21D 13/80; A23G 3/34; A23G 3/48; A23G 3/42; A21D 2/18; A23G 1/36; A23G 1/48; A23G 1/40; A23L 33/125

(52) U.S. Cl. CPC A61K 35/644; A23V 2002/00; A61K 36/88; A61K 36/28; A61K 36/899; A61K 36/9066; A61K 36/54; A61K 36/61; A61K 36/67; A61K 9/0056; A23L 33/115; A23L 33/105; A23L 33/40; A21D 2/165; A21D 2/364; A21D 2/366; A21D 13/28; A21D 13/80; A23G 3/343; A23G 3/48; A23G 3/42; A21D 2/18; A23G 1/36; A23G 1/48; A23G 1/40; A23L 33/125; A61K 36/906

(57) ABSTRACT

A food supplement for helping alleviate the side effects of cancer and cancer treatments is disclosed which is formed from a mixture including at least one edible oil, at least one chopped fruit or fruit paste; at least one ground nut or seed meal or ground nut or seed butter; and honey. The food supplement has a volume of less than three cubic inches and provides at least 100 kilocalories of energy, making it small in volume but with a high energy density. The food supplement can have a high fat content which can make the food supplement moist and easy to eat while providing the necessary calories need for patients combatting cancer and undergoing cancer treatments who often have a severely reduced appetite.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application is a non-provisional of U.S. Patent Application No. 63/034,706 filed Jun. 4, 2020 entitled NUTRITIONAL SUPPLEMENT TO EASE SIDE EFFECTS OF CANCER TREATMENT THERAPIES, which is hereby incorporated by reference in its entireties.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE INVENTION

The present disclosure relates generally to side-effects caused by cancer treatment methods. For example, this document relates to side-effects caused by the toxic nature of chemotherapy and radiation in a disease treatment context.

More particularly, the present disclosure relates to helping remedy the various side-effects of chemotherapy and radiation when used for disease treatment. The American Cancer Society estimates there will be over 1.7 million new cancer cases diagnosed in the United States in 2019. Cancer is the second most common cause of death in the United States with more than 600,000 individuals expected to die from it in 2019. Individuals with cancer are most commonly subject to a treatment method including any combination of surgery, radiation, or chemotherapy. All types of cancer treatment methods produce negative side-effects in patients due to their invasiveness or toxicity. Furthermore, these treatment methods may be used in cases not involving cancer at all, such as the use of chemotherapy to treat blood disorders or autoimmune diseases. When used in other disease contexts, the same negative side-effects are experienced by patients. Chemotherapy can be administered intravenously, topically, by injection, or orally. Chemotherapy generally occurs in rounds or cycles with intervening breaks, and is often completed with multiple combinations of drugs. Chemotherapy fights cancer by targeting and attacking rapidly proliferating cells, regardless of if those cells are cancerous or not. This leads to many negative side effects, especially in areas where rapidly proliferating cells are normal and expected (e.g. hair follicles, digestive tract, and throat/mouth). The main side effect of chemotherapy is a loss of appetite which can be it difficult for the patient to eat and obtain the necessary minimum calories in the several days after treatment, which can exasperate or worsen the other side effects noted herein. Side effects of chemotherapy can also include: fatigue; hair loss; easy bruising and bleeding; infection; anemia; nausea and vomiting; appetite changes; malnutrition; constipation; diarrhea; mouth, tongue, and throat problems such as sores and pain with swallowing caused by inflammation of mucus membranes; numbness, tingling, and pain in nerves and muscles; dry skin and color change in nails; urine and bladder changes; kidney problems; weight changes; changes in concentration and focus (referred to as chemo brain); mood changes; changes in libido and sexual function; fertility problems; and more. Current remedies, including but not limited to medications, liquid protein shakes, supplements, vitamins, and minerals, may only address one or a small subset of these negative and disruptive side effects. Additionally, nutritional supplements currently on the market can be high in calories but can be very difficult for a cancer patient to chew and swallow given the sensitivities of cancer patients after chemotherapy or radiation treatment, particularly those with nausea, mouth sores, sore throats, etc.

What is needed then are improvements to remedies for chemotherapy side-effects in the disease treatment context.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present disclosure provides a food supplement. The single food supplement provided in this disclosure can have a combination of ingredients useful in helping remedy multiple or various side-effects of chemotherapy or radiation in patients while delivering a high caloric and easy to consume food supplement.

The compositions of the present matter provide several specific and new unexpected benefits. The food supplement can help remedy or alleviate one or more negative side-effects of chemotherapy treatment including but not limited to: nausea and vomiting, constipation and diarrhea; urine and bladder changes; weight changes; appetite changes; malnutrition and weight loss; pain with swallowing caused by inflammation of mucus membranes in the throat and mouth; easy bruising and bleeding; infection; fatigue; and chemo brain.

One aspect of the present disclosure is a food supplement formed from a mixture including: at least one edible oil; at least one chopped fruit or fruit paste; at least one ground nut or seed meal or ground nut or seed butter; and honey; wherein the food supplement has a volume of less than three cubic inches and provides at least 100 kilocalories of energy. In some embodiments, the food supplement includes the following percent total weight concentrations: 4% and 25% edible oil; 8% and 22% chopped fruit or fruit paste; 5% and 45% at least one ground nut or seed meal or butter; and 4% and 14% honey.

Another aspect of the disclosure is a food supplement including the following ingredients: an edible oil; a chopped fruit or fruit paste; ginger root or ginger spice; at least one nut or seed meal; and honey. In some embodiments, the food supplement can also include at least one dehydrated fruit powder including a polyphenol; and dehydrated pineapple fruit powder. In some embodiments, the total weight of the food supplement includes between about 15% and 25% edible oil, 8% and 22% chopped fruit, 4% and 10% at least one dehydrated fruit powder including a polyphenol, 5% and 11 ginger root, 5% and 10% dehydrated pineapple fruit powder, 5% and 20% at least one nut or seed meal, and 7% and 14% honey. In some embodiments the food supplement can also include at least about 0.3% tea powder.

In some embodiments the food supplement can include a cake between about 80% and 95% of total weight of said food supplement, the cake including the edible oil; the chopped fruit; the at least one dehydrated fruit powder including a polyphenol, the ginger root, the dehydrated pineapple fruit powder, the at least one nut or seed meal, and the honey; and a frosting between about 6% and 20% of total weight of said food supplement, the frosting including: the at least one dehydrated fruit powder including a polyphenol, the dehydrated pineapple fruit powder, and the honey.

In some embodiments the food supplement can include an edible oil with an energy content of at least 8 kcal/g, the at least one nut or seed meal with an energy content of at least 5 kcal/g, and the chopped fruit with an energy content of at least 3 kcal/g.

In some embodiments the food supplement can include an edible oil with an energy content of at least 6 kcal/g, at least 7 kcal/g, at least 8 kcal/g, or at least 9 kcal/g, the at least one ground nut or seed meal or ground nut or seed butter with an energy content of at least 4 kcal/g, at least 5 kcal/g, at least 6 kcal/g, or at least 7 kcal/g, the chopped fruit with an energy content of at least 2 kcal/g at least 3 kcal/g, or at least 4 kcal/g and a dried, shredded fruit with an energy content of at least 5 kcal/g at least 6 kcal/g, or at least 7 kcal/g.

In some embodiments, the food supplement can further include chocolate, and the food supplement can include a total weight of the food supplement between about: 3% and 10% edible oil; 8% and 22% chopped fruit or fruit paste; 25% and 40% chocolate; 5% and 11° A ginger root; 25% and 45% at least one nut or seed meal; and 6% and 15% honey.

Another aspect of the present disclosure is a method of helping alleviate the side effects of cancer, the method including the steps of: treating a cancer patient with a cancer treatment; prescribing a food supplement be eaten at least three times a day, the food supplement comprising: an edible oil; a chopped fruit or fruit paste; at least one nut or seed meal or butter; and honey; wherein the food supplement has a volume of less than three cubic inches and provides at least 100 kilocalories of energy.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a rendering of the cake and frosting arrangement of the food supplement.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific composition described herein. Such equivalents are considered to be within the scope of this invention and are covered by the claims.

The current invention seeks to help remedy or alleviate multiple side-effects of chemotherapy treatment in a single food supplement. The current invention utilizes a combination of active ingredients which together help reduce inflammation thereby helping remedy soreness when swallowing, diarrhea, urinary and bladder changes, and easy bruising and bleeding; help reduce nausea thereby helping remedy vomiting and appetite changes; contain a high caloric content thereby helping remedy malnutrition, weight changes, fatigue, and nerve and muscle problems; help promote quickened healing of mouth sores; help prevent bacterial and viral infection; and help alleviate the symptoms of chemo brain. This single food supplement can help remedy or alleviate multiple side-effects regularly experienced by patients undergoing chemotherapeutic treatment.

For example, the document provides a food supplement formed from a mixture including: an edible oil; a chopped fruit or fruit paste; ginger root or ginger spice; at least one nut or seed meal; and honey. The food supplement combines multiple ingredients into a mixture which is believed to help reduce multiple side-effects of chemotherapy treatment in a single food supplement. The food supplement is believed to do this through three main categories of symptom relief: anti-nausea, anti-inflammatory, and high nutritional value. Additional symptom relief can include helping remedy chemo brain, promote healing of mouth sores, and help prevent bacterial and viral infection. Certain components of the mixture can help remedy several side-effects from several different categories, and can interact with other components to remedy additional side-effects.

In some embodiments, the food supplement herein can contain at least one dehydrated fruit powder including a polyphenol, which may be a phenolic acid, flavonoid, stilbene and lignan. Polyphenols can support a role in helping prevent degenerative diseases such as cancer through their ability to function as antioxidants. Polyphenols provide a defense against oxidative stress caused by excess reactive oxygen species (ROS), which are regularly formed during chemotherapy treatment and naturally occur in the body. Polyphenols are a numerous and diverse group of natural compounds. Fruits are rich in polyphenols, and contain many different types and classes, including, but not limited to, phenolic acids, flavonoids, stilbenes, and lignans.

Examples of dehydrated fruit powder containing at least one polyphenol contained in the food supplement can include, without limitation, apple, apricot, avocado, banana, bilberry, blackberry, blackcurrant, blood orange, blueberry, cantaloupe, cherry, Chinese date, chokeberry, durian, elderberry, grape, grapefruit, jackfruit, kiwifruit, lemon, mango, navel orange, nectarine, olive, orange, papaya, passionfruit, peach, pear, pineapple, plum, pomegranate, pomelo, prune, raspberry, redcurrant, strawberry, sweetsop and quince.

Examples of polyphenols that can be contained in the at least one dehydrated fruit powder can include, without limitation, benzoic acid, caffeic acid, catechin, cyanidin, epicatechin, ferulic acid, gallic acid, genistein, kaempferol, luteolin, mangiferin, matairesinol, myricetin, naringenin, quercetin, resveratrol, secoisolariciresinol, and taxifolin.

The food supplement can contain any type of a dehydrated fruit powder which can contain any type and number of polyphenols. In addition, the food supplement can contain any amount of the dehydrated fruit powder containing at least one polyphenol. In some embodiments the food supplement provided herein can contain the dehydrated fruit powder containing at least one polyphenol in an amount ranging between about 2% and 10% of the total weight of the food supplement. In some cases, the dehydrated fruit powder containing at least one polyphenol can be provided in an amount between about 2% and 8% of the total weight of the food supplement. In some cases, the dehydrated fruit powder containing at least one polyphenol can be provided in an amount between about 4% and 7% of the total weight of the food supplement.

The food supplement herein can contain a dehydrated pineapple fruit powder that is freeze dried, drum dried, and air dried. Pineapple powder contains a natural enzyme, bromelain, which is commonly used as a phytomedical compound. Bromelain can be absorbed in human intestines without degradation or loss of biological activity, and the human body can absorb a significant amount of bromelain from dehydrated pineapple fruit powder without any major side effects. Bromelain has been recommended to treat chronic inflammatory diseases. The pineapple flavor from the dehydrated pineapple fruit powder can also help provide a pleasant taste for the food supplement.

The food supplement herein can contain any type of a dehydrated pineapple fruit powder. In addition, the food supplement can contain any amount of the dehydrated pineapple fruit powder. In some embodiments, the food supplement herein can contain the dehydrated pineapple fruit powder in an amount ranging between about 2% and 15% of the total weight of the food supplement. In some cases, the dehydrated pineapple fruit powder can be provided in an amount between about 5% and 10% of the total weight of the food supplement. In some cases, the dehydrated pineapple fruit powder can be provided in an amount between about 8% and 15% of the total weight of the food supplement. In some embodiments, the concentration of dehydrated pineapple fruit powder can be greater than or equal to 2, 3, 4, 5, 6, 7, or 8 percent and less than or equal to 10, 11, 12, 13, 14, 15, or 16 percent.

The food supplement herein can contain at least one chopped fruit or fruit paste. Fruits are well-known to have high calorie densities due to their general high sugar content. The high caloric densities of fruits can help remedy the side-effect of weight loss or malnutrition. The chopped fruit may also contain anti-oxidants in the form of polyphenols, which also act as important anti-inflammatory agents. These polyphenols can include: benzoic acid, caffeic acid, catechin, cyanidin, epicatechin, ferulic acid, gallic acid, genistein, kaempferol, luteolin, mangiferin, matairesinol, myricetin, naringenin, quercetin, resveratrol, secoisolariciresinol, and taxifolin.

Examples of fruit that can be used to constitute the chopped fruit or fruit paste contained in the food supplement herein can include, without limitation, one or more of acai, apple, akee, apricot, avocado, banana, berries, bilberry, blackberry, blackcurrant, black sapote, blueberry, blood orange, boysenberry, Buddha's hand, cantaloupe, cherry, cherimoya/custard apple, chico fruit, citrus fruits, clementine, cloudberry, coconut, cranberry, cucumber, crab apples, currant, damson, date, dragonfruit/pitaya, drupes, durian, eggplant, elderberry, feijoa, fig, goja berry, gooseberry, grape, raisin, grapefruit, guava, honeyberry, honeydew, huckleberry, jabuticaba, jackfruit, jambul, Japanese plum, jostaberry, jujube, juniper berry, kiwano/horned melon, kiwifruit, kumquat, lemon, lime, loquat, longan, lychee, mandarin, mango, mangosteen, marionberry, melon, miracle fruit, mulberry, nectarine, nance, orange, olive, papaya, passionfruit, peach, pear, persimmon, plantain, plum, pomes, prune, pineapple, pineberry, plumcot/pluot, pomegranate, pomelo, pumpkin, purple mangosteen, quince, raisins, raspberry, salmonberry, rambutan/mamin chino, redcurrant, salal berry, salak, satsuma, soursop, squash, star apple, star fruit, strawberry, srinam cherry, tamarillo, tamarind, tangerine, tomato, ugli fruit, watermelon, white currant, white sapote, yuzu, and zucchini.

The food supplement herein can contain any type of a chopped fruit or fruit paste. In addition, a food supplement can contain any amount of the chopped fruit or fruit paste. In some embodiments, the food supplement provided herein can contain the chopped fruit or fruit paste in an amount ranging between about 5% and 50% of the total weight of the food supplement. In some cases, the chopped fruit can be provided in an amount between about 5% and 15% of the total weight of the food supplement. In some cases, the chopped fruit can be provided in an amount between about 8% and 12% of the total weight of the food supplement. In some cases, the chopped fruit can be provided in an amount between about 15% and 25% of the total weight of the food supplement. In some cases, the chopped fruit can be provided in an amount between about 35% and 50% of the total weight of the food supplement. In some embodiments, the concentration of the chopped fruit or fruit paste can be greater than or equal to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 percent, and less than or equal to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 percent.

The chopped fruit can contain an energy density of at least 2 kcal/g. In some embodiments, the chopped fruit can contain an energy density of between about 2 kcal/g and 3 kcal/g. In some embodiments, the chopped fruit can contain an energy density of at least 3 kcal/g. In some embodiments, the chopped fruit can contain an energy density of between about 2 kcal/g and 5 kcal/g.

Fruit paste can add the additional advantage as an ingredient of the food supplement of the moisture from the fruit paste helping reduce any chalkiness or dryness within the food supplement, which can make the food supplement more palatable for a patient, and particular for those food supplements including larger quantities of ground powders or meals. Date paste has been found to be a particularly useful as a fruit paste for the supplement as dates are Dates are high in fiber to help with digestion, as well as micronutrients, vitamin C, calcium, iron, phosphorus, potassium, magnesium, and zinc.

The food supplement herein can contain an at least one ground nut or seed meal or ground nut or seed butter which can be a flour or meal that is blanched, gluten-free, brown, white, super-fine ground, fine ground, coarse ground, unbleached, bleached, whole-grain, organic, and high-gluten. Roughly 85% of chemotherapy patients experience malnutrition or weight loss, which can lead to poor outcomes and treatment delays as it becomes harder for the body to rebuild healthy cells without the proper nutrients. Calories and proteins are especially important for individuals undergoing chemotherapy treatments, as they are important for cell repair, tissue healing, fighting infection, and daily activity. Individuals receiving chemotherapy are encouraged to eat more than usual to avoid malnutrition as a result of increased stress on the body. Because chemotherapy also creates the side-effect of decreased appetite, it can be beneficial that individuals eat smaller physical amounts of food with high nutritional value several times per day. Nut and seed meals and butters can provide this type of nutrition in cancer patients, because of the high density amount of calories and proteins they are able to provide.

Examples of nut or seed meal ornut or seed butter that can be used to make the food supplement herein can include, without limitation, almond, buckwheat, cashew, coconut, corn, chestnut, chia, durum, flaxseed, garbanzo fava, gram, green pea, hazelnut, Kyrol, macadamia, millet, peanut, pecan, pistachio, purple corn, quinoa, rice, soy, spelt, sprouted chickpea, sprouted sorghum, sprouted wheat, tapioca, tiger nut, wheat, white, and whole wheat.

The food supplement herein can contain any type of an at least one nut or seed meal or one nut or seed butter. In addition, a food supplement can contain any amount of the at least one nut or seed meal or one nut or seed butter. In some embodiments the food supplement provided herein can contain the at least one nut or seed meal or nut or seed butter in an amount ranging between about 5% and 50% of the total weight of the food supplement. In some cases, the at least one nut or seed meal can be provided in an amount between about 5% and 15% of the total weight of the food supplement. In some cases, the at least one nut or seed meal can be provided in an amount between about 15% and 25% of the total weight of the food supplement. In some embodiments, the at least one nut or seed meal can be provided in an amount between about 25% and 50% of the total weight of the food supplement. In some embodiments, the at least one nut or seed meal can be provided in an amount between about 35% and 45% of the total weight of the food supplement. In some embodiments, the concentration of the at least one nut or seed meal or nut or seed butter can be greater than or equal to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 percent, and less than or equal to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 percent.

The at least one nut or seed meal or nut or seed butter can contain an energy density of at least 3 kcal/g. In some embodiments, the at least one nut or seed meal can contain an energy density of between about 3 kcal/g and 4 kcal/g. In some embodiments, the at least one nut or seed meal or nut or seed butter can contain an energy density of at least 4 kcal/g. In some embodiments, the at least one nut or seed meal or nut or seed butter can contain an energy density of between about 3 kcal/g and 7 kcal/g. In some embodiments, the at least one nut or seed meal or nut or seed butter can contain an energy density of at least about 3 kcal/g, at least about 4 kcal/g, at least about 5 kcal/g, at least about 6 kcal/g, or at least about 7 kcal/g.

The food supplement herein can contain an edible oil, which may be solid or liquid at room temperature. The term “edible oil” includes, without limitation, plant, animal, or synthetic oils which may be saturated, monounsaturated, polyunsaturated, trans-fat, or cis-fat oils. Edible oils have a high fat content, which are an important component of maintaining proper nutrition. The body requires fats to store energy, which is necessary for a body undergoing chemotherapy and increased stress.

Examples of edible oils that can be used to make the food supplement herein can include, without limitation, almond oil, apricot kernel oil, avocado oil, basil oil, brazil nut oil, butter, cashew oil, cocoa butter, coconut oil, corn oil, cottonseed oil, diacyglycerol (DAG) oil, ghee, grapeseed oil, hazelnut oil, hemp oil, lard, linseed oil, macadamia nut oil, margarine, mustard oil, olive oil, palm oil, peanut oil, pumpkin seed oil, rapeseed/canola oil, rice bran oil, safflower oil, sesame oil, soybean oil, sunflower oil, tallow, tea seed oil, vegetable oil, walnut oil, and wheat germ oil. Coconut oil has been found to be particularly beneficial as coconut oil A medium-chain fatty acid that contains lauric acid, an antimicrobial agent. Coconut oil Is considered a heart-healthy, antimicrobial, easily digested fat. This ingredient adds to the soft texture of the food supplement, compliments the flavors of the tropical ingredients, and helps maintain satiety and energy between meals.

The food supplement herein can contain any type of an edible oil. In addition, a food supplement can contain any amount of the edible oil. In some embodiments the food supplement provided herein can contain the edible oil in an amount ranging between about 3% and 35% of the total weight of the food supplement. In some embodiments the food supplement provided herein can contain the edible oil in an amount ranging between about 3% and 10% of the total weight of the food supplement. In some cases, the edible oil can be provided in an amount between about 5% and 15% of the total weight of the food supplement. In some cases, the edible oil can be provided in an amount between about 15% and 20% of the total weight of the food supplement. In some cases, the edible oil can be provided in an amount between about 20% and 35% of the total weight of the food supplement. In some embodiments, the concentration of the at least one edible oil can be greater than or equal to 3, 4, 5, 6, 7, 8, 9, 10, 11, 12, 13, 14, 15, 16, 17, 18, 19, or 20, percent, and less than or equal to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, percent.

The edible oil can contain an energy density of at least 6 kcal/g. In some embodiments, the edible oil can contain an energy density of between about 6 kcal/g and 8 kcal/g. In some embodiments, the edible oil can contain an energy density of at least 8 kcal/g. In some embodiments, the edible oil can contain an energy density of between about 6 kcal/g and 10 kcal/g. In some embodiments, the at least edible oil can contain an energy density of at least about 5 kcal/g, at least about 6 kcal/g, at least about 7 kcal/g, at least about 8 kcal/g, or at least about 9 kcal/g.

The food supplement herein can contain a ginger root or ginger powder, which may be fresh, frozen, dried, powdered, ground, and freeze-dried. Ginger root can be used to treat nausea and vomiting, as well as to stimulate appetite. A ginger supplemented diet can help prevent chemotherapy induced nausea and vomiting in animal models. Furthermore, the chemical compounds [6]-gingerol and [10]-gingerol present in ginger root function to reduce inflammation through inhibiting prostaglandin biosynthesis. Prostaglandins play an important role in the generation of the inflammatory response due to their signaling and activation capabilities. Finally, ginger root contains a very high concentration of anti-oxidants which assist in helping reduce inflammation, which may contribute to pain relief.

Examples of ginger root that can be used to make the food supplement herein can include, without limitation, alpinia, baby, beehive, blue hawaiin, butterfly lily, Chinese/common, culinary, dancing ladies, globba, green, hedychium, hidden, kaempferia, moth, pink, red, shell, small rhizome/Japanese, spiral, spring, stem, torch, white, yellow, young, and zingiber.

The food supplement herein can contain any type of a ginger root or ginger powder. In addition, a food supplement can contain any amount of the ginger root. In some embodiments the food supplement provided herein can contain the ginger root in an amount ranging between about 5% and 15% of the total weight of the food supplement. In some cases, the ginger root can be provided in an amount between about 5% and 7% of the total weight of the food supplement. In some cases, the ginger root can be provided in an amount between about 7% and 11° A of the total weight of the food supplement. In some cases, the ginger root can be provided in an amount between about 11° A and 15% of the total weight of the food supplement. In some embodiments, the concentration of the ginger or ginger root can be greater than or equal to 3, 4, 5, 6, 7, 8, 9, 10 or 11, percent, and less than or equal to 7, 8, 9, 10, 11, 12, 13, 14, 15, 16, 17, 18, 19, or 20 percent. In other cases with certain flavor profiles (chocolate and peanut butter for instance) that aren't cohesive with ginger, the ginger root or ginger powder can be provided in lower weight concentrations, such as between about 0.1% to 3% of the total weight of the food supplement.

The food supplement herein can contain a honey which may be light, amber, and dark as well as raw, organic, unpasteurized, pasteurized, or filtrated. Honey has an array of positive health effects being traditionally used in the treatment of asthma, throat infections, thirst, fatigue, dizziness, constipation, and as a nutrition supplement. Honey can play a role in the treatment of fatigue due to its high calorie density (95%-97% carbohydrate composition) and its ability to provide calories for use in the body immediately after ingestion. Honey can also play an anti-viral role through its ability to stimulate antibody production during primary and secondary immune responses against various antigens. Honey can inhibit infection for almost 60 different types of bacteria including: methicillin-resistant Staphylococcus aureus (MRSA), Escherichia coli, Helicobacter pylori, Bacillus anthracis, Corynebacterium diptheriae, Haemophilus influenza, Lebsiella pneumonia, Listeria monocytogenes, Mycobacterium tuberculosis, Pasteurella multicoda, Yersinia enterocolitica, Proteus species, Pseudomonas aeruginosa, Acinetobacter spp, Salmonella diarrhea, Salmonella typhi, Serratia marcescens, Shigella dysentery, Staphylococcus aureus, Streptococcus faecalis, Streptococcus mutans, Streptococcus pneumonia, Streptococcus pyogenes, and Vibrio cholerae. Unique characteristics of honey, such as high sugar content, low pH, and the presence of hydrogen peroxide, are believed to be the mechanisms through which honey can help inhibit bacterial infection. Honey can also provide a healthy sweetener for the food supplement.

Honey functions as an anti-inflammatory and anti-oxidant, due to the presence of multiple types of polyphenols and other anti-oxidant compounds. Honey can contain flavonoids such as apigenin, pinocembrin, kaempferol, quercetin, galangin, chrysin, and hesperetin; phenolic acids such as ellagic, caffeic, p-coumaric, and ferulic acids; ascorbic acid; tocopherols; catalase; superoxide dismutase; and reduced glutathione.

Examples of honey that can be used to make the food supplement herein can include, without limitation, acacia, alfalfa, avocado, aster, baker's special, basswood, beechwood, blackberry, blueberry, buckwheat, clover, coffee, chestnut, creamed clover, dandelion, eucalyptus, fireweed, golden, heather, iron bark, jarrah, leatherwood, linden, macadamia nut, Manuka, orange blossom, organic, palmetto, premium blend, sage, sourwood, tupelo, wildflower, and yellowbox.

The food supplement herein can contain any type of a honey. In addition, a food supplement can contain any amount of the honey. In some embodiments the food supplement provided herein can contain the honey in an amount ranging from between about 4% and 20% of the total weight of the food supplement. In some cases, the honey can be provided in an amount between about 4% and 8% of the total weight of the food supplement. In some cases, the honey can be provided in an amount between about 8% and 14% of the total weight of the food supplement. In some cases, the honey can be provided in an amount between about 6% and 10% of the total weight of the food supplement. In some embodiments, the concentration of the honey can be greater than or equal to 3, 4, 5, 6, 7, 8, 9, 10, 11, or 12 percent, and less than or equal to 8, 9, 10, 11, 12, 13, 14, 15, 16, 17, 18, 19, or 20 percent.

The food supplement in some embodiments can contain a tea powder which may be black, green, white, oolong, rooibos, and herbal. Tea powder is believed to function as an anti-oxidant and improve memory and cognitive function. Different types of tea powder contain different polyphenols, but they can include: catechins, thearubigins, theaflavins, quercetin, kaempferol, and myricetin.

Examples of tea powder that can be used to make the food supplement provided herein can include, without limitation, amacha, assam, bergamot, catnip, Ceylon, chai, chamomile, cinnamon, dandelion, Darjeeling, Earl Grey, Echinacea, eucalyptus, fennel, feverfew, genmaicha, ginger, ginseng, gunpowder, gyokuro, hawthorn, hibiscus, hojicha, hyssop, jasmine, juniper berry, kabusecha, keemun, lemon, lemongrass, marjoram, matcha, mint, nilgiri, oat straw, oregano, patchouli, rosemary, sencha, Silver Needle, sincha, spearmint, sweet violet, tencha, thyme, uva ursi, valerian, White Peony, yarrow, and yunnan.

The food supplement herein can contain any type of a tea powder. In addition, a food supplement can contain any amount of the tea powder. In some embodiments the food supplement provided herein can contain the tea powder in amount of at least 0.11% of the total weight of the food supplement. In some cases, the tea powder can be provided in an amount between about 0.15% and 0.3% of the total weight of the food supplement. In some cases, the tea powder can be provided in an amount between about 0.3% and 0.55% of the total weight of the food supplement. In some cases, the tea powder can be provided in an amount great than 0.55% of the total weight of the food supplement.

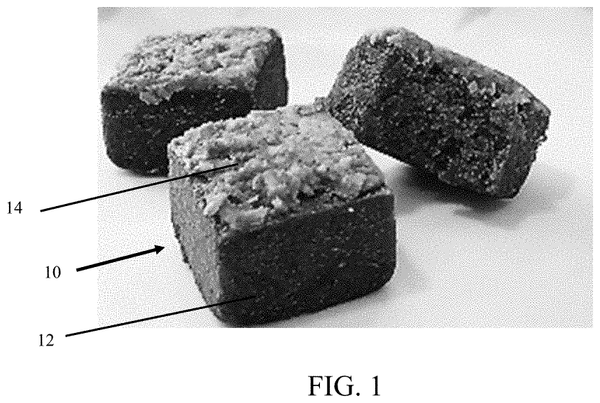

In some embodiments, as shown in FIG. 1, the food supplement 10 can include a cake 12 and a frosting 14. In some cases, the cake 12 can account for an amount ranging between about 75% and 98% of the total weight of said food supplement 10, and the frosting 14 can account for an amount ranging between about 2% and 25% of the total weight of said food supplement 10. In some cases, the cake 12 can be provided in an amount between about 80% and 90% of the total weight of the food supplement 10, and in some cases, the frosting 14 can be provided in an amount between about 10% and 20% of the total weight of the food supplement 10. In some cases, the cake 12 can be provided in an amount between about 92% and 98% of the total weight of the food supplement 10, and in some cases, the frosting 14 can be provided in an amount between about 2% and 8% of the total weight of the food supplement 10.

In some embodiments, the cake 12 can include, without limitation, the edible oil, the chopped fruit, the at least one dehydrated fruit powder including a polyphenol, the ginger root, the dehydrated pineapple fruit powder, the at least one nut or seed meal, and the honey. In some cases, the cake 12 may further include a tea powder. In some embodiments, the frosting 14 can include, without limitation, the at least one dehydrated fruit powder including a polyphenol, the dehydrated pineapple fruit powder, and the honey. In some cases, the frosting 14 may further include a dry, shredded fruit.

In other embodiments, the food supplement 10 can include a mixture of an edible oil; a chopped fruit or fruit paste; ginger root or ginger spice; chocolate; at least one nut or seed meal; and honey. The ingredients previously discussed herein can be included in similar amounts, and the concentration of the chocolate ingredient can be between about 25% and 45%; 25% and 35%; 30% and 40%, or 35% and 45% of the total weight of the food supplement. In some embodiments, the chocolate can have concentrations of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 percent, and less than or equal to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 percent. In some the food supplement can include a total weight of the food supplement between about: 3% and 10% edible oil; 8% and 22% chopped fruit or fruit paste; 30% and 40% chocolate; 0.1% and 5% ginger root or ginger spice; 25% and 45% at least one nut or seed meal or nut or seed butter; and 6% and 15% honey.

In some embodiments, the food supplement can include between about 15 mg and about 25 mg of Cannabidiol (CBD) for the entire food supplement. In some embodiments, the food supplement can include between about 20 mg of CBD. CBD can be included in its raw form or as an oil or cream. CBD can provide a multitude of benefits to chemotherapy patients such as relief from nausea, vomiting, chronic pain, anxiety, insomnia, high blood pressure, etc.

In some embodiments, the food supplement can include a moisture retaining compound such as that sold under the name MoisturLok™ which can be utilized to retain moisture within the food supplement and extend the shelf life of the food supplement. Such a moisture retaining compound can be between about 2% and 4% of the total weight of the food supplement. MoisturLok™ is a fruit based product that provides a sweet taste to food supplements and can function as a binding, humectant, or sweetening agent for the food supplement.

The food supplement can also include various sweetening and/or flavoring components, in addition to the flavorings from the ingredients discussed herein, including but not limited to sugars, spice blends, chamomile powder, vanilla, coconut powder, coconut sugar, salt, etc. In some embodiments, the food supplement further includes a spice blend comprising turmeric, cinnamon, and chamomile, the spice blend having a weight concentration of between 0.3% and 0.1% of the total weight of the food supplement. Turmeric is known for its anti-inflammatory properties, turmeric supports circulation and healing, has a slight bitter detoxifying flavor, offers carminative benefits, has been shown to support the immune system. The bitter detoxifying properties of turmeric are balanced by the sweeter ingredients and balance of additional spices. Cinnamon supports healthy blood sugar metabolism, aids in circulation support, and is antimicrobial. Chamomile provides relief to an overtaxed nervous system, supports digestion by regulating peristalsis, creates a calming effect, and aids in the relief of spasms and pain. In some embodiments, the spice blend further comprises clove and black pepper. Cloves are antimicrobial, promote the flow of digestive juices, aid in respiratory conditions, and can help add a depth and warmth to the flavor profile. Eugenol, the chemical compound in cloves is considered a cancer and tumor inhibitor. Black Pepper is used as a complementary ingredient to the absorption of turmeric. Trace amounts of black pepper can be used such that the flavor profile of this spice is minimal.

The food supplement ingredients can generally be blended together, formed into sheets and can be baked on a low temperature between about 225 and 300 degrees Fahrenheit) for a desired time (between about 60-90 minutes). Once completed, the baked sheet can be cooled and if applicable, a topping or frosting can be applied. The sheet of food supplement can then be cut into bite sized pieces (roughly square or rectangular pieces having a length and width of between about 1-1.5 inches and a depth or height of between about 0.5-1.5 inches. The bite sized food supplements can be have a volume of less than 3 cubic inches, and in some embodiments less than two cubic inches. Depending on the chosen ingredients, a package of three bite sized food supplements can provide between about 250 and 400 kilocalories. In some embodiments, the package of three bite sized food supplements can provide between about 300-350 kilocalories. As such, each bite sized piece having a volume of less than 3 cubic inches, or in some embodiments, two cubic inches, can offer at least 100 kilo calories per bite-sized food supplement. In some embodiments, each bite-sized food supplement can offer at least 130 kilocalories per bite sized food supplement.

Another unique aspect of the food supplement of the present disclosure is its high fat content. In some embodiments, the food supplement can include at least 15, 16, 17, 18, 19, 20, 21, 22, 23, 24, or 25 grams of fat. The food supplement can include at least 3, 4, 5, 6, 7, 8, 9, 10, 11, or 12 grams of protein to provide a more protein rich food supplement. However the high fat content of the food supplement of the present disclosure allows for the food supplement to maintain a moist and soft consistency, with a “melt-in-your mouth” consistency, which can allow for the food supplement to be more easily chewed and/or swallowed. The high fat and moist characteristics of the food supplement are particularly useful for patients suffering from nausea, mouth sores, sore throats, who may have a hard time chewing and swallowing a more dry, chalky, chewy, high protein, low fat supplement which is typical in the nutritional bar and supplement field. The food supplement can also include at least 20, 21, 22, 23, 24, 25, 26, 27, 28, 29, or 30 grams of carbohydrates to help provide an energy dense food supplement.

Another aspect of the present disclosure is a method of helping alleviate the side effects of cancer, the method including the steps of: treating a cancer patient with a cancer treatment; prescribing a food supplement be eaten at least three times a day, the food supplement comprising: an edible oil; a chopped fruit or fruit paste; at least one nut or seed meal or butter; and honey; wherein the food supplement has a volume of less than three cubic inches and provides at least 100 kilocalories of energy. Prescribing at least 3 of the food supplements be consumed daily can help ensure the patients calorie intake is sufficient to fight the cancer and overcome the negative side effects of the cancer treatment. In practice, the food supplements of the present disclosure would be prescribed to a cancer patient after treatment to supplement the patient's other medications and provide a high caloric supplement to help prevent malnutrition. The food supplements of the present disclosure could be included in the medical insurance systems and medical insurance coverages and prescribed as a treatment protocol for cancer patients alongside their radiation, chemotherapy, immunotherapies, etc.

Additionally, it has been found that taste changes are a common side effect of many chemotherapy regimens. Patients typical report that after chemotherapy treatments, spicy, sweet, and sour flavor profiles are generally tolerable depending on the type of treatment received or cancer of the patient. The food supplements in some embodiments can include large amounts of ginger and other spices to create a spicy flavor profile, with chocolate, peanut butter, and chamomile to create a sweet flavor profile, and with cherries, lemons and other sour fruits to achieve the sour profile. Packages of food supplements can come in packages of three bite sized food supplements either all in one flavor or a combination of the three for variety and to receive the various medical and healing benefits of the different flavor profile and food supplement formulations and ingredients.

Thus, although there have been described particular embodiments of the present invention of a new and useful NUTRITIONAL SUPPLEMENT TO EASE SIDE EFFECTS OF CANCER TREATMENT THERAPIES, it is not intended that such references be construed as limitations upon the scope of this invention.

What is claimed is:

1. A food supplement formed from a mixture comprising:
at least one edible oil;
at least one chopped fruit or fruit paste;
at least one ground nut or seed meal or ground nut or seed butter; and
honey;
wherein the food supplement has a volume of less than three cubic inches and provides at least 100 kilocalories of energy.

2. The food supplement of claim 1, further comprising:
ginger or ginger root;
at least one dehydrated fruit powder including a polyphenol; and
dehydrated pineapple fruit powder.

3. The food supplement of claim 2, wherein said food supplement comprises the following percent total weight concentrations:
15% and 25% edible oil;
8% and 22% chopped fruit or fruit paste;
4% and 10% at least one dehydrated fruit powder including a polyphenol;
5% and 11% ginger root or ginger spice;
5% and 10% dehydrated pineapple fruit powder;
5% and 20% ground nut or seed meal or ground nut or seed butter; and 7% and 14% honey.

4. The food supplement of claim 2, wherein the food supplement comprises:
a cake between about 80% and 95% of total weight of said food supplement, the cake comprising: the at least one edible oil, the at least one chopped fruit, the at least one dehydrated fruit powder including a polyphenol, the ginger or ginger root, the dehydrated pineapple fruit powder, the at least one ground nut or seed meal or ground nut or seed butter, and the honey; and
a frosting between about 5% and 20% of total weight of said food supplement, the frosting comprising: the at least one dehydrated fruit powder including a polyphenol, the dehydrated pineapple fruit powder, and the honey.

5. The food supplement of claim 4, wherein:
the frosting further comprises a dried, shredded fruit with an energy content of at least 6 kcal/g.

6. The food supplement of claim 1, wherein:
the at least one edible oil has an energy content of at least 8 kcal/g;
the at least one ground nut or seed meal or ground nut or seed butter has an energy content of at least 5 kcal/g; and
the at least one chopped fruit has an energy content of at least 3 kcal/g.

7. The food supplement of claim 1, wherein:
the at least one ground nut or seed meal or ground nut or seed butter has a protein percent weight relative to a total weight of the at least one ground nut or seed meal or ground nut or seed butter between about 20% and 25%.

8. The food supplement of claim 1, wherein:
the at least one chopped fruit has a carbohydrate percent weight relative to a total weight of the chopped fruit between about 75% and 95%.

9. The food supplement of claim 1, wherein:
The at least one edible oil has a fat percent weight relative to a total weight of the edible oil between about 98% and 99%.

10. The food supplement of claim 1, further comprising:
a tea powder comprising at least 0.1% total weight of the food supplement wherein the tea powder comprises chamomile powder and/or oat straw tea powder.

11. The food supplement of claim 1, further comprising:
chocolate, wherein the food supplement comprises a total weight of the food supplement of between about:
3% and 10% edible oil;
8% and 22% chopped fruit or fruit paste;
25% and 40% chocolate;
30% and 45% at least one nut or seed meal; and
4% and 15% honey.

12. A food supplement comprising:
a volume of less than 3.3 cubic inches;
wherein said food supplement comprises the following percent total weight concentrations:
4% and 25% edible oil;
8% and 22% chopped fruit or fruit paste;
5% and 45% at least one ground nut or seed meal or butter; and
4% and 14% honey; and
wherein the food supplement provides at least 100 kilocalories per supplement.

13. The food supplement of claim 12, wherein the food supplement provides at least 20 grams of fat, at least 25 grams of carbohydrates, and at least 3 grams of protein per supplement.

14. The food supplement of claim 12, further comprising:
between 35% and 45% at least one ground nut or seed butter;
between 30% and 38% dairy-free chocolate;
between 0.1% and 0.3% chamomile and/or oat straw tea powder; and
between 8% and 15% of a fruit paste;
wherein the food supplement provides at least 25 grams of fat, at least 35 grams of carbohydrates, and at least 10 grams of protein per supplement, and at least 130 total kilocalories per supplement.

15. The food supplement of claim 12, where the food supplement has a volume of less than 2 cubic inches.

16. The food supplement of claim 12, wherein the food supplement further comprises a spice blend comprising turmeric, cinnamon, and chamomile, the spice blend having a weight concentration of between 0.3% and 0.1% of the total weight of the food supplement.

17. The food supplement of claim 16, wherein the spice blend further comprises clove and black pepper.

18. The food supplement of claim 12, further comprising:
15% and 25% edible oil;
12% and 20% chopped fruit or fruit paste;
4% and 10% at least one dehydrated fruit powder including a polyphenol;
6% and 10% ginger root or ginger spice;
5% and 10% dehydrated pineapple fruit powder
5% and 20% at least one ground nut or seed meal or butter; and
9% and 11% honey.

19. A method of helping alleviate the side effects of cancer, the method comprising the steps of:
treating a cancer patient with a cancer treatment;
prescribing a food supplement be eaten at least three times a day, the food supplement comprising:
an edible oil;
a chopped fruit or fruit paste;
at least one nut or seed meal or butter; and
honey;
wherein the food supplement has a volume of less than three cubic inches and provides at least 100 kilocalories of energy.

20. The method of claim 19, wherein the food supplement has at least 20 grams of fat, 25 grams of carbohydrates, and 3 grams of protein.
